(19) United States
(12) Patent Application Publication
(10) Pub. No.: US 2017/0164934 A1
(43) Pub. Date: Jun. 15, 2017

(54) METHOD AND DEVICE FOR EXTRACTING MULTIPLE TISSUE SAMPLES

(71) Applicant: Vanderbilt University, Nashville, TN (US)

(72) Inventors: Bryan Hartley, Nashville, TN (US); Philip J. Swaney, Nashville, TN (US); Robert J. Webster, Nashville, TN (US); Lars Axel Strombergsson, Nashville, TN (US)

(21) Appl. No.: 15/386,170

(22) Filed: Dec. 21, 2016

Related U.S. Application Data
(63) Continuation-in-part of application No. 15/349,737, filed on Nov. 11, 2016.
(60) Provisional application No. 62/253,881, filed on Nov. 11, 2015.

(51) Int. Cl. A61B 10/02

(52) U.S. Cl. CPC A61B 10/02; A61B 10/0275; A61B 2010/0208; A61B 2010/0225

(57) ABSTRACT

A multipurpose tissue sampling device is provided for extracting at least one tissue sample from a tissue sampling site. An outer sheath has a cutting surface and an outer sheath lumen extending from an outer sheath proximal end to an outer sheath distal end. An inner stylet has a solid inner stylet cutting tip, sampling notch, and an inner stylet lumen extending from an inner stylet proximal end to the sampling notch. The inner stylet exterior wall is configured to pass through the outer sheath lumen. A fine needle aspiration needle has an FNA needle body configured to pass through the inner stylet lumen. The FNA needle has an FNA needle lumen and a sampling feature. An actuator has a proximal port and a distal port with inner port walls that are configured to accept at least a portion of at least one of the outer sheath and inner stylet.

### RELATED APPLICATION

This application is a continuation-in-part of U.S. patent application Ser. No. 15/349,737, filed 11 Nov. 2016, and claiming priority from U.S. Provisional Application No. 62/253,881, filed 11 Nov. 2015, the subject matter of all of which is incorporated herein by reference in its entirety.

### GOVERNMENT SUPPORT

This invention was made with United States government support under contract R21 EB017952 awarded by the National Institutes of Health. The government has certain rights in the invention.

### TECHNICAL FIELD

This disclosure relates to an apparatus and method for use of a multipurpose tissue sampling device and, more particularly, to a method and device for extracting multiple tissue samples.

### BACKGROUND

Accurately diagnosing small pulmonary nodules may provide a diagnostic challenge for physicians, as many of the nodules are benign and may require no intervention. However, some nodules may be hard to identify as needing treatment because they appear on a computed tomography (“CT”) scan to be no different than benign nodules. In order to determine whether certain nodules are problematic, a physician may perform a tissue biopsy.

Biopsy needle position accuracy is important in extracting a desired sample (diagnostic accuracy) and reducing potential complications that follow a biopsy. Diagnostic accuracy becomes more difficult the smaller the nodule is. For example, according to some sources, when categorized for nodule size, overall, 73.5% of nodules greater than 1.5 cm yielded a diagnostic specimen. On the other hand, only 51.4% of nodules less than or equal to 1.5 cm yielded a diagnostic specimen. Biopsy needle position accuracy may also prevent oversampling, the extraction of tissue beyond the nodule, which can lead to such complications as hemorrhaging and pneumothorax. For various reasons, including avoidance of patient discomfort, many small nodules are not biopsied. Instead, these small nodules are watched, potentially delaying treatment and allowing small, localized malignancies to advance.

### SUMMARY

In an aspect, a multipurpose tissue sampling device is provided. The multipurpose tissue sampling device includes an outer sheath, an inner stylet, a fine needle aspiration (“FNA”) needle, and an actuator. The outer sheath has an outer sheath body and an outer sheath distal end. The outer sheath distal end has an outer sheath cutting surface. The outer sheath body defines an outer sheath lumen. The inner stylet has an inner stylet body and an inner stylet distal end. The inner stylet has a sampling notch positioned on at least one of the inner stylet body and the inner stylet distal end. The inner stylet body is configured to pass through the outer sheath lumen. The inner stylet has an inner stylet lumen that extends between the inner stylet body and the sampling notch. The FNA needle has an FNA needle body and a FNA needle distal end. The FNA needle body is configured to pass through the inner stylet lumen. The FNA needle body defines a FNA needle lumen. The FNA needle distal end has a sampling feature. The actuator is configured to accept a portion of at least one of the outer sheath, the inner stylet, and the FNA needle.

In an aspect, a method for extracting at least one tissue sample from a tissue sampling site is provided. A multipurpose tissue sampling device is provided. The multipurpose tissue sampling device includes an outer sheath, an inner stylet, a fine needle aspiration (“FNA”) needle, and an actuator. The outer sheath has an outer sheath body and an outer sheath distal end. The outer sheath distal end has an outer sheath cutting surface. The outer sheath body defines an outer sheath lumen. The inner stylet has an inner stylet body and an inner stylet distal end. The inner stylet has a sampling notch positioned on at least one of the inner stylet body and the inner stylet distal end. The inner stylet body is configured to pass through the outer sheath lumen. The inner stylet has an inner stylet lumen that extends between the inner stylet body and the sampling notch. The FNA needle has an FNA needle body and a FNA needle distal end. The FNA needle body is configured to pass through the inner stylet lumen. The FNA needle body defines a FNA needle lumen. The FNA needle distal end has a sampling feature. The actuator is configured to accept a portion of at least one of the outer sheath, the inner stylet, and the FNA needle. The outer sheath is attached to the actuator. The inner stylet is inserted through the actuator and into the outer sheath lumen. The multipurpose tissue sampling device is inserted into a desired patient tissue sampling site with the multipurpose tissue sampling device in an inserting position. The multipurpose tissue sampling device is actuated into a sampling position by pulling the outer sheath proximally. The FNA needle is inserted into the inner stylet lumen. A suction means is attached to the FNA needle. At least a portion of the desired patient tissue is suctioned from the patient tissue sampling site into the sampling notch of the multipurpose tissue sampling device via the suction means. The FNA needle is further inserted into the inner stylet lumen until the FNA needle contacts the desired patient tissue that was suctioned into the sampling notch. An FNA sample is obtained by at least one of shaving and cutting the desired patient tissue that was suctioned into the sampling notch of the inner stylet with the FNA needle sampling feature. The FNA needle, with the included FNA sample, is removed from the inner stylet lumen. A core tissue sample is obtained by actuating the multipurpose tissue sampling device to transition from the sampling position to the inserting position, wherein as the multipurpose tissue sampling device transitions from the sampling position to the inserting position, the core tissue sample is severed by the outer sheath cutting surface. The multipurpose tissue sampling device, with the included core tissue sample, is removed from the desired patient tissue sampling site.

In an aspect, a multipurpose tissue sampling device is provided. The multipurpose tissue sampling device includes an outer sheath, an inner stylet, a fine needle aspiration (“FNA”) needle, and an actuator. The outer sheath has an outer sheath proximal end and an outer sheath distal end. The outer sheath proximal and distal ends are separated longitudinally by an outer sheath body. The outer sheath proximal end has an outer sheath attachment hub. The outer sheath distal end has an outer sheath cutting surface. The outer sheath body has an outer sheath exterior wall. The outer sheath has an outer sheath lumen that extends between the outer sheath proximal end and the outer sheath distal end. The outer sheath lumen has an outer sheath lumen wall. The inner stylet has an inner stylet proximal end and an inner stylet distal end. The inner stylet proximal and distal ends are separated longitudinally by an inner stylet body. The inner stylet distal end has a solid inner stylet cutting tip. The sampling notch is longitudinally spaced from the inner stylet cutting tip. The sampling notch is positioned on at least one of the inner stylet body and the inner stylet distal end. The inner stylet body has an inner stylet exterior wall configured to pass through the outer sheath lumen. The inner stylet has an inner stylet lumen that extends between the inner stylet proximal end and the sampling notch. The inner stylet lumen has an inner stylet lumen wall. The FNA needle has an FNA needle proximal end and an FNA needle distal end separated longitudinally by an FNA needle body. The FNA needle body is configured to pass through the inner stylet lumen. The FNA needle body has an FNA needle lumen that extends between the FNA needle proximal end and the FNA needle distal end. The FNA needle distal end has a sampling feature. The actuator has an actuator proximal end and an actuator distal end separated longitudinally by an actuator body. The actuator proximal end has a proximal port. The actuator distal end has a distal port. The proximal and distal ports each have an inner port wall that is configured to accept at least a portion of at least one of the outer sheath and inner stylet.

### BRIEF DESCRIPTION OF THE DRAWINGS

For a better understanding, reference may be made to the accompanying drawings, in which:

FIG. 1 is a front view of a multipurpose tissue sampling device according to one aspect of the present invention in a first position;

FIG. 2 is a front view of the multipurpose tissue sampling device of FIG. 1 in a second position;

FIG. 3 is a side view of an element of the aspect of FIG. 1;

FIG. 4 is a side view of another element of the aspect of FIG. 1;

FIG. 5 is a partial cross-sectional view of another element of the aspect of FIG. 1;

FIG. 6 is an partial cross-sectional view of an element of the aspect of FIG. 1;

FIG. 7 is a rear view of an element of the aspect of FIG. 1;

FIGS. 8A-F illustrate an example sequence of operation of a portion of the aspect of FIG. 1;

FIGS. 9A-E illustrate an example sequence of operation of a portion of the aspect of FIG. 1;

FIG. 10 is a front view of a multipurpose tissue sampling device of the aspect of FIG. 1 in an alternate configuration; and

FIG. 11 is a front view of a multipurpose tissue sampling device of the aspect of FIG. 1 in an alternate configuration.

### DESCRIPTION OF ASPECTS OF THE DISCLOSURE

Unless defined otherwise, all technical and scientific terms used herein have the same meaning as is commonly understood by one of skill in the art to which the present disclosure pertains.

As used herein, the term “patient” can refer to any warm-blooded organism including, but not limited to, human beings, pigs, rats, mice, dogs, goats, sheep, horses, monkeys, apes, rabbits, cattle, farm animals, livestock, etc.

As used herein, the term “user” can be used interchangeably to refer to an individual who prepares for, assists, and/or performs a procedure.

As used herein, the singular forms “a,” “an” and “the” can include the plural forms as well, unless the context clearly indicates otherwise. It will be further understood that the terms “comprises” and/or “comprising,” as used herein, can specify the presence of stated features, steps, operations, elements, and/or components, but do not preclude the presence or addition of one or more other features, steps, operations, elements, components, and/or groups thereof.

As used herein, the term “and/or” can include any and all combinations of one or more of the associated listed items.

As used herein, phrases such as “between X and Y” and “between about X and Y” can be interpreted to include X and Y.

As used herein, phrases such as “between about X and Y” can mean “between about X and about Y.”

As used herein, phrases such as “from about X to Y” can mean “from about X to about Y.”

It will be understood that when an element is referred to as being “on,” “attached” to, “connected” to, “coupled” with, “contacting,” etc., another element, it can be directly on, attached to, connected to, coupled with or contacting the other element or intervening elements may also be present. In contrast, when an element is referred to as being, for example, “directly on,” “directly attached” to, “directly connected” to, “directly coupled” with or “directly contacting” another element, there are no intervening elements present. It will also be appreciated by those of skill in the art that references to a structure or feature that is disposed “directly adjacent” another feature may have portions that overlap or underlie the adjacent feature, whereas a structure or feature that is disposed “adjacent” another feature may not have portions that overlap or underlie the adjacent feature.

Spatially relative terms, such as “under,” “below,” “lower,” “over,” “upper” and the like, may be used herein for ease of description to describe one element or feature's relationship to another element(s) or feature(s) as illustrated in the Figures. It will be understood that the spatially relative terms can encompass different orientations of a device in use or operation, in addition to the orientation depicted in the Figures. For example, if a device in the Figures is inverted, elements described as “under” or “beneath” other elements or features would then be oriented “over” the other elements or features.

It will be understood that, although the terms “first,” “second,” etc. may be used herein to describe various elements, these elements should not be limited by these terms. These terms are only used to distinguish one element from another. Thus, a “first” element discussed below could also be termed a “second” element without departing from the teachings of the present disclosure. The sequence of operations (or steps) is not limited to the order presented in the claims or Figures unless specifically indicated otherwise.

The invention comprises, consists of, or consists essentially of the following features, in any combination.

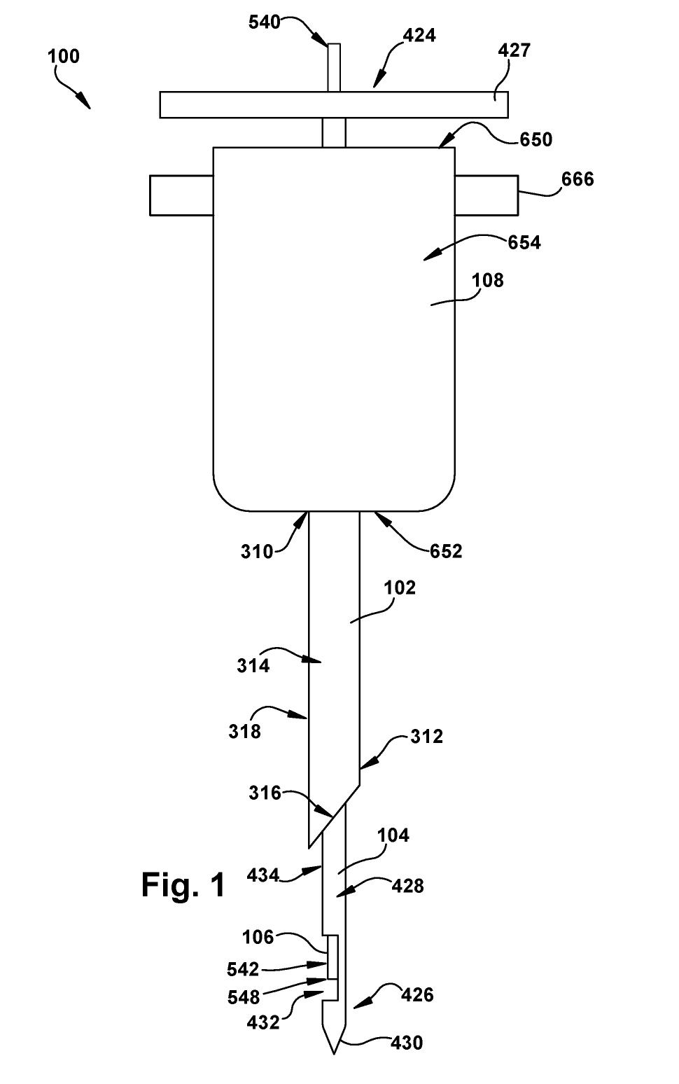

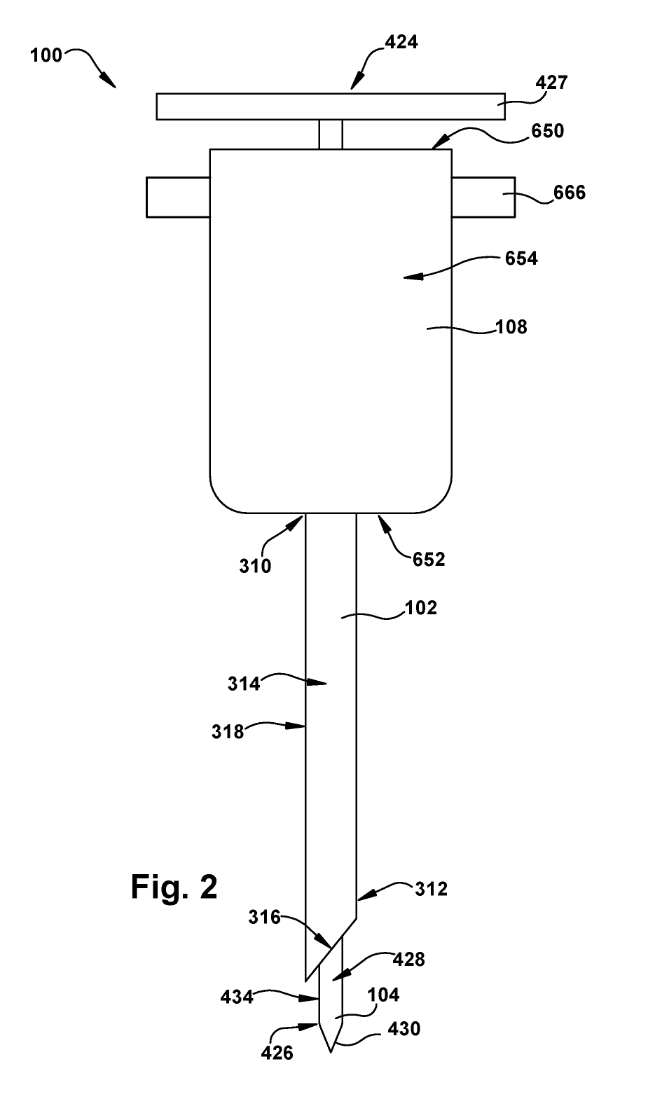

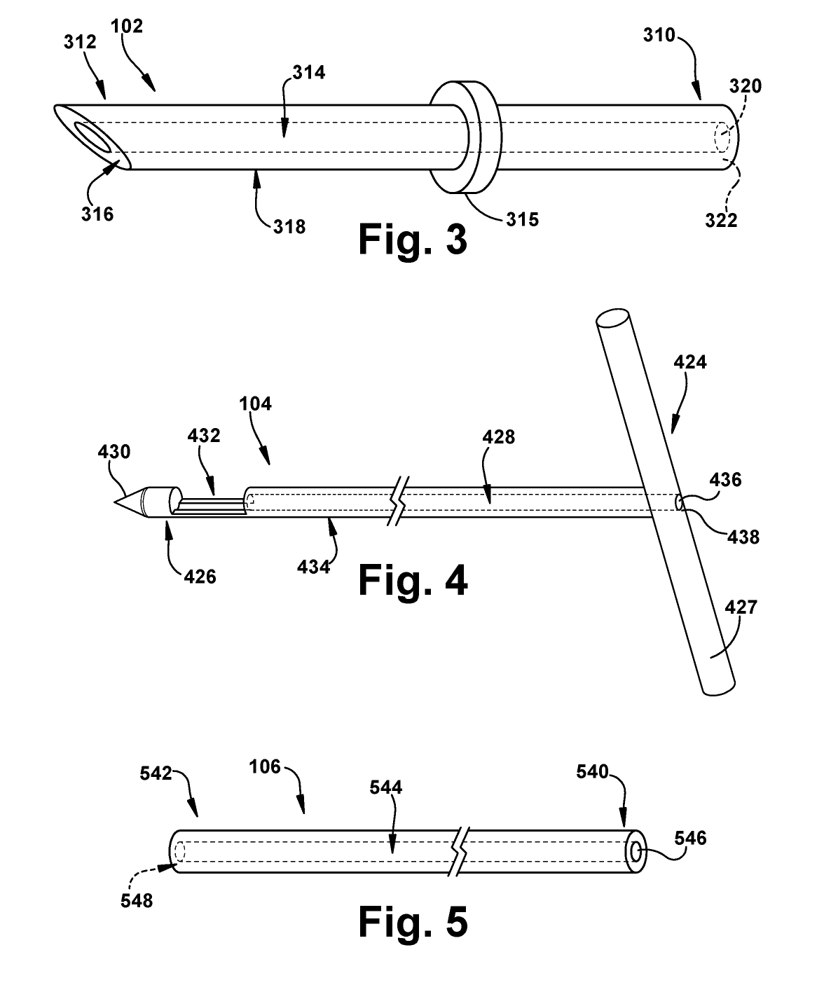

FIGS. 1-2 depict a multipurpose tissue sampling device 100 including an outer sheath 102, an inner stylet 104, a fine needle aspiration (“FNA”) needle 106, and an actuator 108. As shown in FIG. 3, the outer sheath 102 has an outer sheath proximal end 310 and an outer sheath distal end 312. The outer sheath proximal and distal ends 310, 312 are separated longitudinally by an outer sheath body 314. The term “longitudinally” is used herein to indicate a substantially vertical direction, in the orientation of FIGS. 1-2. The outer sheath body 314, although depicted in the Figures as having a cylindrical shape, can have any suitable shape for taking biopsy samples.

The outer sheath proximal end 310 has an outer sheath attachment hub 315, which attaches the outer sheath 102 to at least one of the inner stylet 104, the FNA needle 106, and the actuator 108. The outer sheath proximal end 310 may have finger grips (not shown). The outer sheath distal end 312 has an outer sheath cutting surface 316. The outer sheath cutting surface 316 can be, but is not limited to, a single- or multi-beveled cutting surface. The outer sheath body 314 has an outer sheath exterior wall 318. The outer sheath 102 has an outer sheath lumen 320 extending longitudinally between the outer sheath proximal end 304 and the outer sheath distal end 306. The outer sheath lumen 320 has an outer sheath lumen wall 322.

As shown in FIG. 4, the inner stylet 104 has an inner stylet proximal end 424 and an inner stylet distal end 426. An inner stylet handle 427 may be disposed on the inner stylet proximal end 424. The inner stylet handle 427 may be a permanent feature of the inner stylet 104, an attachable/detachable feature, or any combination thereof. The inner stylet proximal and distal ends 424, 426 are separated longitudinally by an inner stylet body 428. The inner stylet body 428, although shown in the Figures as having a cylindrical shape, may have any suitable shape for taking biopsy samples. The inner stylet distal end 426 has a solid inner stylet cutting tip 430. The inner stylet cutting tip 430 can be, but is not limited to, a single- or multi-beveled tip. The inner stylet body 428 has a sampling notch 432. The sampling notch 432 is longitudinally spaced from the inner stylet cutting tip 430. The sampling notch 432 is positioned on at least one of the inner stylet body 432 and the inner stylet distal end 426. Although the sampling notch 432 is shown as being rectangular in shape, it is contemplated that the sampling notch 432 may have any suitable shape such as, but not limited to, circular, oval-like, and saddle-like. The solid inner stylet cutting tip 430 restricts the FNA needle 106 from protruding from the sampling notch 432 and sampling the benign tissue outside of a desired patient tissue sampling site T.

The inner stylet body 428 has an inner stylet exterior wall 434 which is configured to pass through the outer sheath lumen 320. For example, the inner stylet exterior wall 434 may be smaller in diameter than the outer sheath lumen wall 322. The inner stylet 104 has an inner stylet lumen 436 extending between the inner stylet proximal end 424 and the sampling notch 432. The sampling notch 432 places the inner stylet exterior wall 434 in fluid connection with the inner stylet lumen 436. The inner style lumen 436 has an inner stylet lumen wall 438. The inner stylet 104 may have an attachment mechanism (not shown) for attaching to at least one of the outer sheath 102, the FNA needle 106, and the actuator 108.

As shown in FIG. 5, the FNA needle 106 has an FNA needle proximal end 540 and an FNA needle distal end 542. The FNA needle proximal end 540 may be configured to accept the attachment of any desired accessory item thereto, such as a suction/vacuum source, a fluid source, or any other suitable surgical aid. The FNA proximal and distal ends 540, 542 are separated longitudinally by an FNA needle body 544. The FNA needle body 544 is configured to pass through the inner stylet lumen 436. For example, the FNA needle body 544 may be smaller in diameter than the inner stylet lumen wall 438. The FNA needle body 544, although shown to have a cylindrical shape in the Figures, can have any suitable shape for taking FNA samples. The FNA needle body 544 has an FNA needle lumen 546 that extends between the FNA needle proximal end 540 and the FNA needle distal end 542. The FNA needle distal end 542 has a sampling feature 548. The sampling feature 548 may be of any suitable type, such as the sampling features of commercially available FNA needles (not shown). The FNA needle 106 may have an attachment mechanism (not shown) for attaching to at least one of the outer sheath 102, inner stylet 104, and actuator 108.

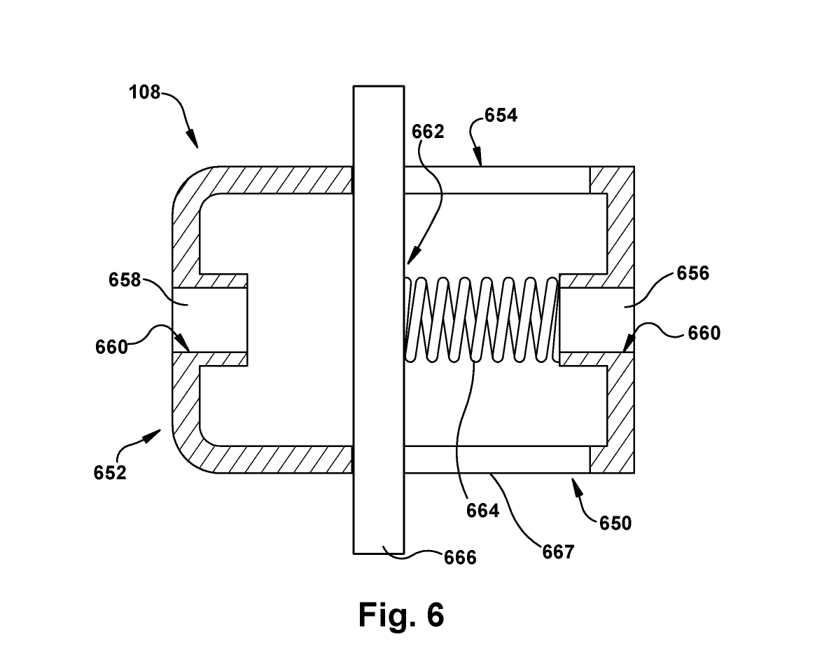

As shown in FIG. 6, the actuator 108 has an actuator proximal end 650 and an actuator distal end 652. The actuator proximal and distal ends 650, 652 are separated longitudinally by an actuator body 654. The actuator proximal end 650 has a proximal port 656. The actuator distal end 652 has a distal port 658. The proximal and distal ports 656, 658 each have an inner port wall 660 configured to accept a portion of at least one of the outer sheath 102 and inner stylet 104. As discussed below, the distal port 658 may extend at least a portion in the proximal direction. The actuator 108 may be configured to accept the attachment means of at least one of the inner stylet 104 and FNA needle 106.

The actuator 108 may also include a firing mechanism 662. The firing mechanism 662 has at least one biasing element 664, at least one triggering element 666, and at least one locking mechanism 667. The biasing element 664 can be any appropriate biasing element, such as a spring. The triggering element 666 may be configured to accept at least a portion of the outer sheath 102. The triggering element 666 may have finger grips (shown in FIG. 11 as 1174). The triggering element 666 may be directly attached to at least one of the outer sheath 102 and the outer sheath attachment hub 315.

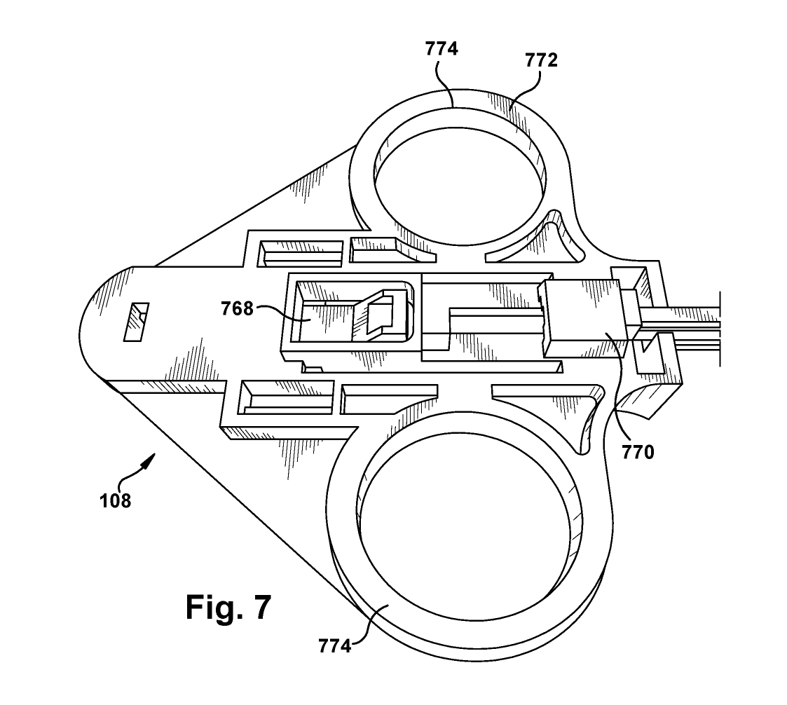

An example of the locking mechanism 667 is shown in FIG. 7. The locking mechanism 667 can include a catch mechanism 768 that will be configured to accept a portion of the outer sheath attachment hub 315 to restrict distal movement of the outer sheath 102. The locking mechanism 667 further includes a release mechanism 770. The release mechanism 770 is configured to unlock the catch mechanism 768, thereby facilitating distal movement of the outer sheath 102. Further, as can be seen in FIG. 7, the actuator may include an actuator handle 772 and actuator finger grips 774.

In use, the multipurpose tissue sampling device 100, as described above, is provided to the user. The user attaches the outer sheath 102 to the actuator 108. The user may insert the inner stylet 104 through the actuator 108 and into the outer sheath lumen 320. The user locates a desired patient tissue sampling site T through the use of any suitable means, such as a CT scanner.

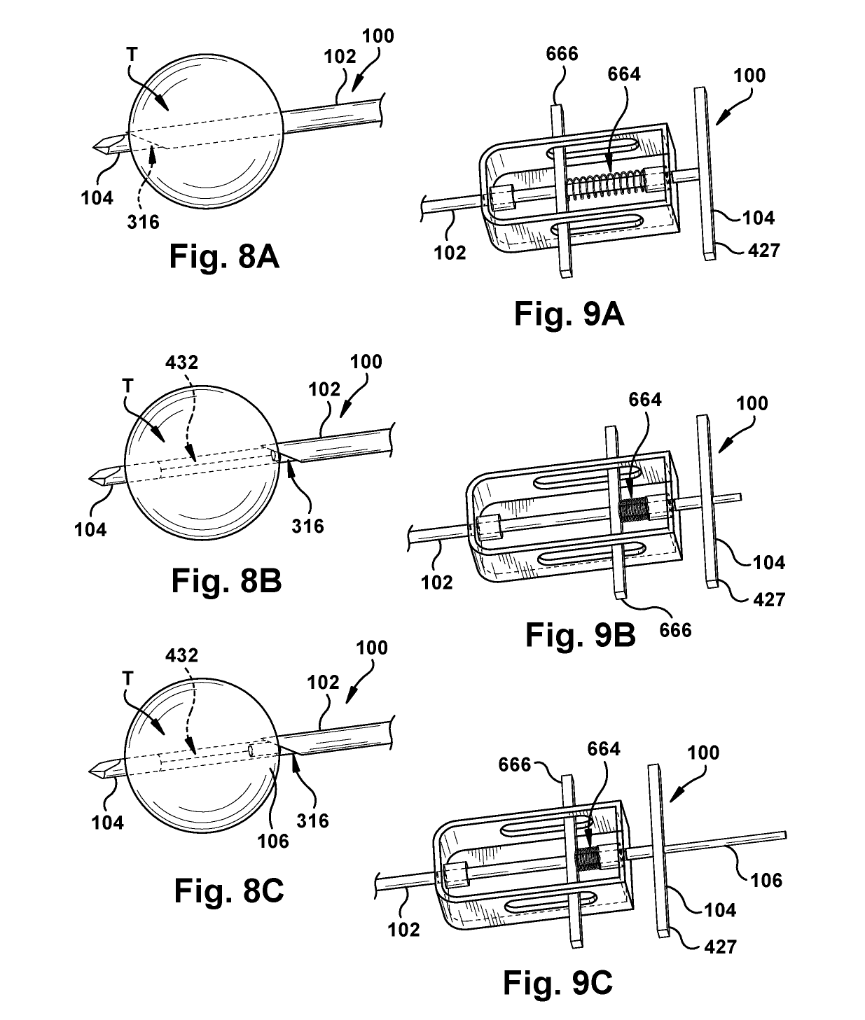

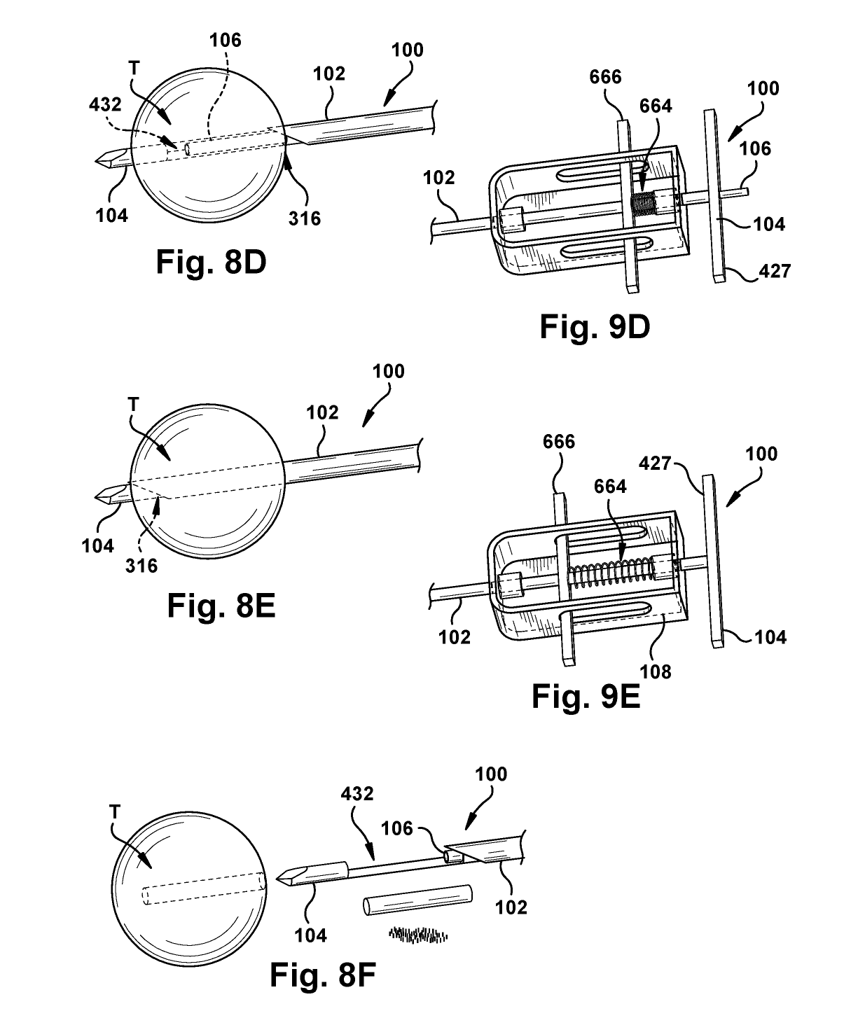

As shown in FIG. 8A, the user inserts the multipurpose tissue sampling device 100 into the desired patient tissue sampling site T with the multipurpose tissue sampling device 100 in an inserting position. FIG. 2 more clearly shows the multipurpose tissue sampling device 100 in the inserting position. When the multipurpose tissue sampling device 100 is in the inserting position, the sampling notch 432 is covered by the outer sheath lumen wall 322 while the inner stylet cutting tip 430 protrudes from the outer sheath distal end 312, as shown in FIG. 2. The inserting position allows the user to insert the multipurpose tissue sampling device 100 into the desired patient tissue sampling site T while restricting any unwanted tissue from entering the sampling notch 432 from the side. The user can selectively place the multipurpose tissue sampling device 100 in the inserting position by moving the outer sheath 102 distally over the inner stylet 104 until the outer sheath lumen wall 322 covers the sampling notch 432.

As shown in FIG. 8B, the user actuates the multipurpose tissue sampling device 100 into a sampling position by pulling the outer sheath 102 proximally a desired, but adjustable, distance to expose a predetermined amount of the sampling notch 432. This transition, as shown in FIGS. 9A-9B, can be accomplished by attaching the outer sheath to the triggering element 666, moving the triggering element 666, with attached outer sheath 102, in the proximal direction (toward the right, in the direction of FIGS. 9A-9B), which compresses the biasing element 664, and locking the triggering element 666 to restrict undesired movement of the outer sheath 102. FIG. 1 more clearly shows the multipurpose tissue sampling device 100 in the sampling position. When the multipurpose tissue sampling device 100 is in the sampling position, the outer sheath lumen wall 322 does not cover at least a portion of the sampling notch 432, thereby exposing at least a portion of the sampling notch 432 to the desired patient tissue sampling site T.

If the user wants to take an FNA sample from the desired patient tissue sampling site T, the user inserts the FNA needle 106 into the inner stylet lumen 436 (shown in FIGS. 8C and 9C). A suction means is attached to the FNA needle proximal end 540. The user suctions at least a portion of the desired patient tissue at the desired patient tissue sampling site T into the sampling notch 432 via the suction means.

As shown in FIGS. 8D and 9D, the user further inserts the FNA needle 106 into the inner stylet lumen 436 until the FNA needle 106 contacts the desired patient tissue that was suctioned into the sampling notch 432. An FNA sample is then obtained by shaving and/or cutting the desired patient tissue that was suctioned into the sampling notch 432 of the inner stylet 104 with the sampling feature 548 of the FNA needle 106. Once the sample is obtained, the user can remove the FNA needle 106, with the included FNA sample, from the inner stylet lumen 436 and the multipurpose tissue sampling device 100, while maintaining multipurpose tissue sampling device 100 in the desired patient tissue sampling site T. Because the multipurpose tissue sampling device 100 is maintained in the desired patient tissue sampling site T, if the user desires to take multiple FNA samples, the user can then reinsert the FNA needle 106 (or another FNA needle, not shown) into the inner stylet lumen 436 and repeat the process, described above, as desired.

The multipurpose tissue sampling device 100 may be rotated within the desired patient tissue sampling site T following the removal of the FNA needle 106, in order to gain access to patient tissue that has not already been sampled by the FNA needle, before obtaining the core tissue sample. The user can obtain a core tissue sample by actuating the multipurpose tissue sampling device 100 to transition from the sampling position to the inserting position, wherein when the multipurpose tissue sampling device 100 transitions from the sampling position to the inserting position, the core tissue sample (which was previously suctioned into the sampling notch 432) is severed by the outer sheath cutting surface 316. For example, the user can unlock the triggering element 666 which causes the biasing element 664 within the actuator 108 to uncompress. Uncompressing the biasing element 664 causes the triggering element 666, with attached outer sheath 102, to move from the sampling position to the inserting position, as shown in FIG. 8E and 9E.

Depending upon factors including the strength of the biasing element 664, this movement of the triggering element 666 and outer sheath 102 between the sampling position and the inserting position could be quite rapid (a “snapping” motion). The multipurpose tissue sampling device 100, or components thereof, should be configured, for many use environments, to “trigger” and drive motion of the outer sheath 102 into the inserting position with sufficient force to sever the core tissue sample in a desired manner. As shown in FIG. 8F, the user can then remove the multipurpose tissue sampling device 100 with the included severed tissue sample from the desired patient tissue sampling site T.

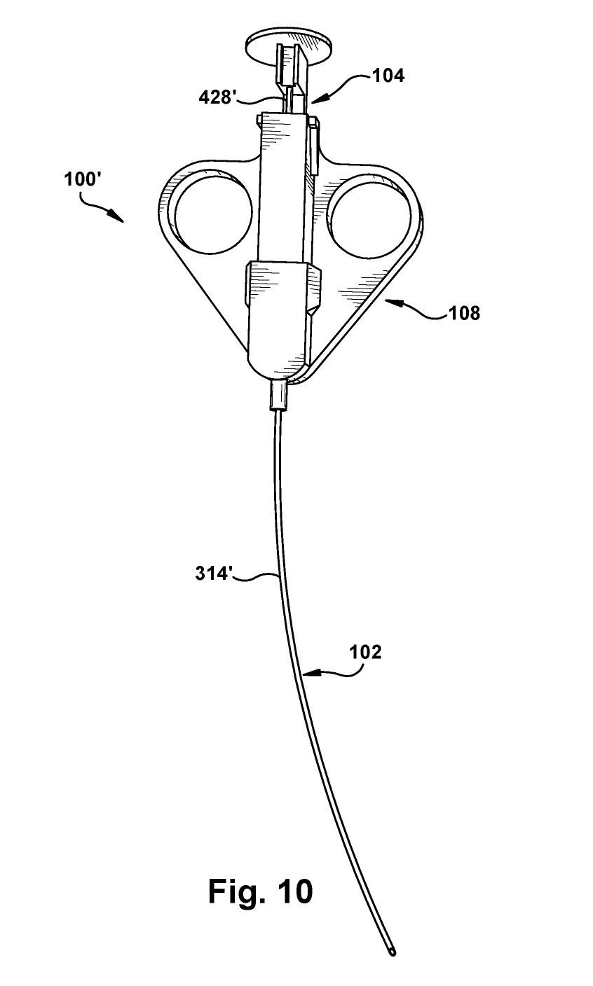

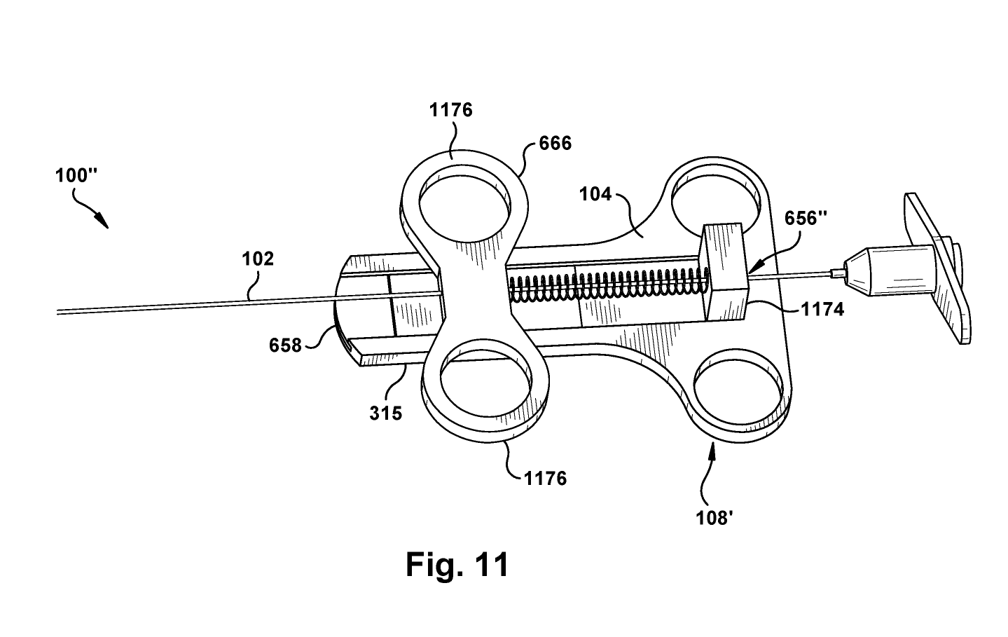

FIG. 10 illustrates a second configuration of the multipurpose tissue sampling device 100′, which may differ from that shown in FIGS. 1-6. Therefore, structures of FIG. 10 that are the same as or similar to those described with reference to FIGS. 1-6 are either unnumbered or have the same reference numbers with the addition of a “prime” mark. Description of common elements and operation similar to those in the previously described first configuration will not be repeated with respect to the second configuration, for brevity.

The main distinction of the multipurpose tissue sampling device 100′ of the second configuration from that of the multipurpose tissue sampling device 100 of the first configuration is the shape of at least one of the outer sheath body 314, inner stylet body 428, and FNA needle body 544. For example, as shown in FIG. 10, the multipurpose tissue sampling device 100′ of the second configuration may have a curved outer sheath body 314′. The curved outer sheath body 314′ allows the multipurpose tissue sampling device 100′ to access desired tissue sampling sites which may be difficult to reach using the straight-line access path of the outer sheath body 314 of the first configuration, as shown in FIG. 3.

The multipurpose tissue sampling device 100′ of the second configuration may have a curved inner stylet body 428′, as shown in FIG. 10. The curved inner stylet body 428′ allows the multipurpose tissue sampling device 100′ to access desired tissue sampling sites which may be inaccessible by the straight-line access path of the inner stylet body 428. The curved inner stylet body 428′ may be elastic. This elastic configuration allows the curved inner stylet body 428′ to first follow a straight path through the outer sheath body 314 of FIG. 4 and then follow a curved path upon exiting the outer sheath lumen 320.

The multipurpose tissue sampling device 100′ may have a curved FNA needle body (not shown). The curved FNA needle body allows the multipurpose tissue sampling device 100′ to access desired tissue sampling sites which may be inaccessible by the straight-line access path of the FNA needle body 544. The curved FNA needle body may be elastic. This elastic configuration allows the curved FNA needle body to first follow a straight path through the outer sheath body 314 of FIG. 3 and then follow the curved trajectory of the curved inner stylet body 428′ of FIG. 10.

FIG. 11 illustrates a third configuration of the multipurpose tissue sampling device 100″, which may differ from that shown in FIGS. 1-10. Therefore, structures of FIG. 11 that are the same as or similar to those described with reference to FIGS. 1-10 are either unnumbered or have the same reference numbers with the addition of a “double-prime” mark. Description of common elements and operation similar to those in the previously described first and second configurations will not be repeated with respect to the third configuration, for brevity.

The main distinction of the multipurpose tissue sampling device 100″ of the second configuration from that of the multipurpose tissue sampling device 100 of the first configuration is the addition of an inner stylet attachment hub 1174. As shown in FIG. 11, the inner stylet 104 is attached to an inner stylet attachment hub 1174, which is attached to the actuator 108″. In this configuration, the inner stylet attachment hub 1174 acts as the actuator's 108″ proximal port 656″. Further, the biasing element extends between the triggering element 666 and the inner stylet attachment hub 1174.

FIG. 11 shows, as discussed above, that the triggering element 666 may be configured to accept the user's fingers, such as through the use of finger grips 1176, and that the triggering element 666 may also be directly attached to the outer sheath attachment hub 315. Further, FIG. 11 shows, as discussed above, the distal port 658 extending at least a portion in the proximal direction.

Although shown in the Figures as being solid, it is contemplated that the inner stylet cutting tip 430 of any configuration of the multipurpose tissue sampling device 100 may include a lumen (not shown) extending the inner stylet lumen 436 to from the inner stylet proximal end 424 and entirely through the inner stylet cutting tip 430.

The outer sheath 102, the inner stylet 104, the FNA needle 106, and/or the actuator can each be at least partially formed from silicone, polyethylene, polypropylene, stainless steel, titanium, any other biocompatible material, or any combination thereof. The outer sheath 102, the inner stylet 104, the FNA needle 106, and/or the actuator can each be at least partially formed from magnetic resonance imaging (“MRI”) and/or CT compatible materials such as, but not limited to, polyetheretherketone, nonmetal polymers, nonmetal ceramics, any other nonmetal material, or any combination thereof. The use of MRI and/or CT compatible materials may assist the user in reducing imaging artifacts on MRI and/or CT images that may be caused by using biopsy tools that contain metal. The term “artifact” is defined herein as a structure that is not anatomically present in a MRI and/or CT image, but is visible in the MRI and/or CT image.

While aspects of this disclosure have been particularly shown and described with reference to the example aspects above, it will be understood by those of ordinary skill in the art that various additional aspects may be contemplated. For example, the specific methods described above for using the apparatus are merely illustrative; one of ordinary skill in the art could readily determine any number of tools, sequences of steps, or other means/options for placing the above-described apparatus, or components thereof, into positions substantively similar to those shown and described herein. In an effort to maintain clarity in the Figures, certain ones of duplicative components shown have not been specifically numbered, but one of ordinary skill in the art will realize, based upon the components that were numbered, the element numbers which should be associated with the unnumbered components; no differentiation between similar components is intended or implied solely by the presence or absence of an element number in the Figures. Any of the described structures and components could be integrally formed as a single unitary or monolithic piece or made up of separate sub-components, with either of these formations involving any suitable stock or bespoke components and/or any suitable material or combinations of materials; however, the chosen material(s) should be biocompatible for many applications. Any of the described structures and components could be disposable or reusable as desired for a particular use environment. Any component could be provided with a user-perceptible marking to indicate a material, configuration, at least one dimension, or the like pertaining to that component, the user-perceptible marking potentially aiding a user in selecting one component from an array of similar components for a particular use environment. A “predetermined” status may be determined at any time before the structures being manipulated actually reach that status, the “predetermination” being made as late as immediately before the structure achieves the predetermined status. The term “substantially” is used herein to indicate a quality that is largely, but not necessarily wholly, that which is specified—a “substantial” quality admits of the potential for some relatively minor inclusion of a non-quality item. Though certain components described herein are shown as having specific geometric shapes, all structures of this disclosure may have any suitable shapes, sizes, configurations, relative relationships, cross-sectional areas, or any other physical characteristics as desirable for a particular application. Any structures or features described with reference to one aspect or configuration could be provided, singly or in combination with other structures or features, to any other aspect or configuration, as it would be impractical to describe each of the aspects and configurations discussed herein as having all of the options discussed with respect to all of the other aspects and configurations. A device or method incorporating any of these features should be understood to fall under the scope of this disclosure as determined based upon the claims below and any equivalents thereof.

Other aspects, objects, and advantages can be obtained from a study of the drawings, the disclosure, and the appended claims.

We claim:

1. A multipurpose tissue sampling device, comprising:
an outer sheath, the outer sheath having an outer sheath body and an outer sheath distal end, the outer sheath distal end having an outer sheath cutting surface, the outer sheath body defining an outer sheath lumen;
an inner stylet, the inner stylet having an inner stylet body and an inner stylet distal end, the inner stylet having a sampling notch positioned on at least one of the inner stylet body and the inner stylet distal end, the inner stylet body being configured to pass through the outer sheath lumen, the inner stylet having an inner stylet lumen extending between the inner stylet body and the sampling notch;
a fine needle aspiration (“FNA”) needle, the FNA needle having an FNA needle body and a FNA needle distal end, the FNA needle body being configured to pass through the inner stylet lumen, the FNA needle body defining a FNA needle lumen, the FNA needle distal end having a sampling feature; and
an actuator being configured to accept a portion of at least one of the outer sheath, the inner stylet, and the FNA needle.

2. The multipurpose tissue sampling device of claim 1, wherein the actuator includes a firing mechanism, the firing mechanism having at least one biasing element, at least one triggering element, and at least one locking mechanism.

3. The multipurpose tissue sampling device of claim 2, wherein the locking mechanism includes a catch mechanism, the catch mechanism being configured to accept at least a portion of the outer sheath body to restrict distal movement of the outer sheath.

4. The multipurpose tissue sampling device of claim 3, wherein the locking mechanism includes a release mechanism, the release mechanism being configured to unlock the catch mechanism, thereby facilitating distal movement of the outer sheath.

5. The multipurpose tissue sampling device of claim 1, including the inner stylet body being smaller in diameter than the outer sheath lumen, and the FNA needle body being smaller in diameter than the inner stylet lumen.

6. The multipurpose tissue sampling device of claim 1, wherein at least one of the outer sheath body, inner stylet body, and FNA needle body is curved.

7. The multipurpose tissue sampling device of claim 1, wherein the outer sheath, the inner stylet, the FNA needle, and the actuator are at least partially formed from a nonmetal material.

8. The multipurpose tissue sampling device of claim 1, wherein the inner stylet distal end has a solid inner stylet cutting tip 430 that restricts the FNA needle from protruding from the sampling notch.

9. A method for extracting at least one tissue sample from a tissue sampling site, the method comprising:
providing a multipurpose tissue sampling device including;
attaching the outer sheath to the actuator;
inserting the inner stylet through the actuator and into the outer sheath lumen;
inserting the multipurpose tissue sampling device into a desired patient tissue sampling site with the multipurpose tissue sampling device in an inserting position;
actuating the multipurpose tissue sampling device into a sampling position by pulling the outer sheath proximally;
inserting the FNA needle into the inner stylet lumen;
attaching a suction means to the FNA needle proximal end;
suctioning at least a portion of the desired patient tissue from the tissue sampling site into the sampling notch of the multipurpose tissue sampling device via the suction means;
further inserting the FNA needle into the inner stylet lumen until the FNA needle contacts the desired patient tissue that was suctioned into the sampling notch;
obtaining an FNA sample by at least one of shaving and cutting the desired patient tissue that was suctioned into the sampling notch with the FNA needle sampling feature;
removing the FNA needle with the included FNA sample from the inner stylet lumen;
obtaining a core tissue sample by actuating the multipurpose tissue sampling device to transition from the sampling position to the inserting position, wherein when the multipurpose tissue sampling device transitions from the sampling position to the inserting position, the core tissue sample is severed by the outer sheath cutting surface; and
removing the multipurpose tissue sampling device, with the included severed tissue sample, from the tissue sampling site.

10. The method of claim 9, including maintaining multipurpose tissue sampling device 100 in the desired patient tissue sampling site after the FNA needle is removed from the inner stylet lumen.

11. The method of claim 9, including:
selectively moving the outer sheath distally over the inner stylet until the outer sheath lumen covers the sampling notch;
wherein when the sampling notch is covered by the outer sheath lumen, the multipurpose tissue sampling device is in the inserting position.

12. The method of claim 11, including:
pulling the outer sheath proximally a desired, but adjustable, distance to expose a predetermined amount of the sampling notch;
wherein when a predetermined amount of the sampling notch is exposed, the multipurpose tissue sampling device is in the sampling position.

13. The method of claim 12, including:
providing an actuator having a firing mechanism, the firing mechanism having at least one biasing element, at least one triggering element, and at least one locking mechanism.
attaching the outer sheath to the triggering element;
moving the triggering element, with the attached outer sheath, in the proximal direction to compress the biasing element, thereby placing the multipurpose in the sampling position; and
locking the triggering element to restrict undesired movement of the outer sheath.

14. The method of claim 13, including:
unlocking the triggering element; and
uncompressing the biasing element;
wherein the uncompressing biasing element causes the triggering element, with attached outer sheath, to move from the sampling position to the inserting position.

15. A multipurpose tissue sampling device, comprising:
an outer sheath, the outer sheath having an outer sheath proximal end and an outer sheath distal end, the outer sheath proximal and distal ends being separated longitudinally by an outer sheath body, the outer sheath proximal end having an outer sheath attachment hub, the outer sheath distal end having an outer sheath cutting surface, the outer sheath body having an outer sheath exterior wall, the outer sheath having an outer sheath lumen extending between the outer sheath proximal end and the outer sheath distal end, the outer sheath lumen having an outer sheath lumen wall;
an inner stylet, the inner stylet having an inner stylet proximal end and an inner stylet distal end, the inner stylet proximal and distal ends being separated longitudinally by a inner stylet body, the inner stylet distal end having a solid inner stylet cutting tip, the inner stylet body having a sampling notch, the sampling notch being longitudinally spaced from the inner stylet cutting tip, the sampling notch being positioned on at least one of the inner stylet body and the inner stylet distal end, the inner stylet body having an inner stylet exterior wall, the inner stylet exterior wall being configured to pass through the outer sheath lumen, the inner stylet having an inner stylet lumen extending between the inner stylet proximal end and the sampling notch, the inner stylet lumen having an inner stylet lumen wall;
a fine needle aspiration (“FNA”) needle, the FNA needle having an FNA needle proximal end and an FNA needle distal end separated longitudinally by an FNA needle body, the FNA needle body being configured to pass through the inner stylet lumen, the FNA needle body having an FNA needle lumen extending between the FNA needle proximal end and the FNA needle distal end, the FNA needle distal end having a sampling feature; and
an actuator having an actuator proximal end and an actuator distal end separated longitudinally by an actuator body, the actuator proximal end having a proximal port, the actuator distal end having a distal port, the proximal and distal ports each having an inner port wall configured to accept a portion of at least one of the outer sheath, the inner stylet, and the FNA needle.

16. The multipurpose tissue sampling device of claim 13, wherein the actuator includes a firing mechanism, the firing mechanism having at least one biasing element, at least one triggering element, and at least one locking mechanism.

17. The multipurpose tissue sampling device of claim 14, wherein the locking mechanism includes a catch mechanism, the catch mechanism being configured to accept at least a portion of the outer sheath attachment hub to restrict distal movement of the outer sheath.

18. The multipurpose tissue sampling device of claim 15, wherein the locking mechanism includes a release mechanism, the release mechanism being configured to unlock the catch mechanism, thereby facilitating distal movement of the outer sheath.

19. The multipurpose tissue sampling device of claim 13, including the inner stylet exterior wall 434 being smaller in diameter than the outer sheath lumen wall, and the FNA needle body being smaller in diameter than the inner stylet lumen wall.

20. The multipurpose tissue sampling device of claim 13, wherein at least one of the outer sheath body, inner stylet body, and FNA needle body is curved.
